(19) United States
(12) Patent Application Publication
(10) Pub. No.: US 2007/0110674 A1
(43) Pub. Date: May 17, 2007

(54) SONO-ACTIVE LIPOSOMES AND LIPID PARTICLES AND USE THEREOF AS CONTRAST AGENTS AND ACTIVE-AGENT DELIVERY SYSTEMS

(76) Inventors: Yuhong Xu, Shanghai (CN); Xiaohui Wei, Shanghai (CN)

Correspondence Address: ROSENBAUM & ASSOCIATES, 650 DUNDEE ROAD, SUITE 380, NORTHBROOK, IL, 60062-2757, US

(21) Appl. No.: 11/460,958

(22) Filed: Jul. 28, 2006

Related U.S. Application Data
(60) Provisional application No. 60/704,106, filed on Jul. 29, 2005.

(51) Int. Cl. A61K 49/22; A61K 9/127; C12N 15/88; A61K 48/00

(52) U.S. Cl. 424/9.51; 424/450; 435/458; 977/929

(57) ABSTRACT

The present invention is directed to a novel class of gas filled liposomal delivery system particles, and to methods of making these particles, including the production of gas filled liposomal delivery system particles via chemical reaction to produce gas bubbles inside the particles. The present invention also includes: gas filled liposomal delivery system particles containing one or more active agents; gas filled liposomal delivery system particles for visualization purposes, e.g., for visualization of structures of the body via ultrasound waves; gas filled liposomal delivery system particles for the targeted delivery of active agent to one or more areas of interest of the body; and, gas filled liposomal delivery system particles for the targeted delivery of active agent to one or more areas of interest of the body, where targeting specificity is enhanced by active targeting to direct the gas filled liposomal delivery system particles to a particular target.

### CROSS-REFERENCE TO RELATED APPLICATIONS

The present application relates to and claims priority to co-pending provisional application, Ser. No. 60/704,106, filed Jan. 29, 2005.

### BACKGROUND OF THE INVENTION

Microbubbles, with the sizes of few micrometers or even nanometers, can effectively scatter ultrasound waves because of the large acoustic impedance differences between the gas space and the surrounding liquid. They are useful both in diagnostic and therapeutic applications. Early in the 1960s, scientists employed rudimentary ultrasound contrast agents (UCAs). In the next forty years, UCAs formulations progressed rapidly from free-gas bubbles (agitated saline and ECHOVIST) to the second and third generation encapsulated bubbles.

ALBUNEX™, the first agent approved by the US FDA in 1994, utilized sonicated human serum albumin to encapsulate and stabilize air microbubbles. LEVOVIST™ was generated by shaking small galactose granules and 0.1% palmitic acid in saline solution. But the control of size and stability after injection limit their clinical applications. However, liposomes offer significant improvements. SONOVUE™, the third generation UCAs, consists of phospholipid-stabilized microbubbles filled with sulfur hexafluoride (SF₆) with outstanding stability and resistance to pressure. For the last decade, many scientists hammered at developing new UCAs with improved techniques. Wheatley and Singhal obtained stable microbubbles (called ST68) filled with air by sonication of mixtures of nonionic surfactants. Researchers at Northwestern Universtiy prepared air-filled liposomes by a dehydration/rehydration method.

Gas-containing particles with specific acoustical reflection characteristics for ultrasound wave are now widely used in the diagnostic ultrasound imaging. With the development of in situ localization of ultrasound, focused ultrasound-guided in vivo delivery of therapeutic agents will be a promising delivery system with high specificity and accuracy. Reproducible and controllable preparation methods for making gas-containing acoustically reflecting particles capable of drug-loading is, therefore, critical.

Denatured albumin, liposome and lipid based micelle are commonly adopted as vehicles for the encapsulation and stabilization for gas bubbles inside. Some has entered clinical use as ultrasound contrast enhancement agents, including SONOVUE™, INFINITY™ AND LEVOVIST™. Gases such as air or fluorocarbons are encapsulated into the intravesicular space through a pressure decrease method, physical stirring method, acid-base reaction and heat or photon catalyst method. The pressure decrease method, physical stirring method and acid-base reaction are easier to operate but the gas encapsulation process is difficult to control. Further loading of therapeutic drugs onto these gas containing vehicles would require more complex procedures such as chemical conjugation. Therefore, it is necessary for a new preparation method development and optimization of gas encapsulation and drug loading processes.

The delivery of various active agents into the specific diseased tissue in the body is of vital importance to the diagnosis, prevention, and treatment of disease. In terms of diagnosis of disease, for example, the diagnostic efficiencies of techniques such as ultrasound imaging (USI) and magnetic resonance imaging (MRI) are markedly improved by delivery of contrast agents, such as acoustically active or gadolinium-based contrast agents, to the area of the body to be imaged. Similarly, disease prevention or treatment methods critically depend upon the action of one or more active agents delivered into the diseased site, e.g., the action of an anti-tumor drug delivered to a region of cancer in the body, etc.

A particularly effective approach to the delivery of active agents into the body is using a “liposomal” delivery system (“LDS”), i.e., a system in which the active agent or active agents to be delivered are encapsulated in lipid-based vesicles. Such LDSs include the following three structural categories of liposomes: unilamellar liposomes, or, synonymously, unilamellar vesicles (“ULVs”); multilamellar lipsomes, or, synonymously, multilamellar vesicles (“MLVs”); multivesicular liposomes (“MVLs”); and other lipid membrane enclosed complex particles (“lipoplexes”). In this regard, UVLs may be defined as liposomes where a single internal aqueous compartment is enclosed; MLVs may be defined as containing multiple concentric chambers within each liposome particle resembling the layers of an onion; and, MVLs may be defined as liposomes containing multiple non-concentric chambers within each liposome particle. See, e.g., U.S. Pat. No. 5,807,572, the contents of which are herein incorporated by reference in their entirety.

Active agents for diagnosis, prevention, or therapeutic purposes can be loaded onto the LDSs using various methods described in the literature. These agents can be made associated with IDSs by physical encapsulation, hydrogen bonding, hydrophobic interaction, and/or electrostatic interactions. For example, hydrophilic drug molecules can be loaded into the internal aqueous space of a liposome by physical encapsulation. Hydrophobic drugs can be incorporated in the lipid membrane. Biopolymers, such as DNA and RNA, may be complexed with oppositely charged liposomes to form lipoplexes. Lipid compositions in the various LDSs can be optimized for desirable structure and drug loading capacities.

The LDSs of various compositions may have specific in vivo distribution properties based on their physico-chemical properties, which would in the end result in different active agent delivery patterns. For example, some large particles (˜micron in diameter) are easily recognized and cleared by the reticuloendothelial system (RES) after iv injection. Smaller particles, especially those with surface PEG modification have prolonged half-lives in circulation and more passes through the leaky tumor vasculature. The so-called EPR (enhanced permeability and retention) effect would then promote the extravasation and accumulation of LDSs that are smaller than the pore size of tumor endothelia. In other applications, “active targeting” strategies have been developed to attach targeting antibodies or ligands to LDSs that would interact with specific molecular target in the diseased site for more focused delivery.

One category of LDS of particular interest are echogenic or acoustically active lipid delivery systems (“AALDS”), where the liposomes or other lipid based particles contain a gas that can be used to aid in visualization of the AALDS particles in the body, and that may additionally be used to control delivery of an active agent or agents from the AALDS particles. Such gas microbubbles can aide in the visualization of the AALDS particles in the body by, for example, ultrasound waves, which will be scattered by the gas microbubbles. The gas microbubbles in a AALDS may also be used as a control system for releasing an active agent or agents also encapsulated in the AALDS particles, for example by the application of ultrasound pulses to rupture the AALDS particles (via cavitation of the gas bubbles) when they have reached the target area for active agent-release.

Although AALDSs have great promise for active-agent delivery, to date methods of preparation of these systems have been problematic. Preparation of AALDS particles has been based primarily on: shaking methods; sonication methods; and, dehydration/rehydration methods. Each of these methods has various limitations, including, for example, limitations resulting from the comparatively large sized AALDS particles produced for sufficient gas encapsulation. Specifically, due to the micron size of typical formulations of AALDS particles (i.e., easily taken up by the reticuloendothelial system), typical AALDS preparations are not well suited for, e.g., intravascular administration, or penetration into smaller anatomical spaces.

One particular important application in using acoustically active delivery system is to deliver genes (or other nucleic acid based therapeutics) to specific cells in the body. Ultrasound energy alone can increase cell membrane permeability and thereby facilitate gene transfection. Ultrasound-mediated destruction of microbubbles can enhance this effect further by the mechanism of acoustic cavitation.

Therefore acoustically active LDSs could be extremely useful by carrying genes to the desired site, and then releasing as well as delivering the gene to the target cells by directed ultrasound irradiation. One of the most commonly used gene delivery LDS in the area is cationic lipid/DNA complexes (lipoplexes). But it has been hard to entrap air inside such lipoplexes in the preparation to make them acoustically active. There have been only studies using commercial microbubble contrast agents such as OPTISON™, ALBUNEX™ and BR14. But since all these agents were not designed for or even capable of carrying drugs especially DNA molecules, they'd have limited applications.

In light of the preceding, there is therefore a need for new methods for making sono-active liposomes such as AALDSs, including AALDSs of a size range better suited to entry into the areas of the body largely inaccessible to conventional AALDSs, e.g., AALDSs based on “nano sized liposomes or lipid particles and vesicles.” Such novel AALDSs would have unique utilities, for example in extravasation through the vasculature of tumors, for tumor imaging, drug-delivery to tumors, and targeted gene therapy etc.

### SUMMARY OF THE INVENTION

An embodiment method for preparation of gas containing liposomes and lipid particles based on chemical reactions in intravesicular space, comprising preparation of the liposome or lipid particle encapsulating one or more reactants, and then introducing the other reactants by membrane controlled diffusion to generate gas bubbles inside liposomes.

Another embodiment of the invention is a method for preparation of AALDS, comprising preparation of liposome or lipid particle encapsulated with insoluble gas forming precursors or soluble reactants that would interact over time or upon light or heat stimulation inside the liposomes.

Another embodiment of the present invention is to attach antibody fragment, peptide or small molecule ligands to the surface of the AALDS by incorporating into the lipid membrane.

Another embodiment of the present invention is directed to the AALDS particles of the invention containing one or more therapeutic drugs/gene constructs.

Another aspect of the present invention is directed to the use of the AALDS particles of the present invention for visualization purposes, e.g., for visualization of structures of the body via ultrasound waves.

Another embodiment of the present invention is directed to the use of the AALDSs of the present invention in the targeted delivery of active agent to one or more areas of interest of the body. In one aspect of this embodiment, AALDS particles containing at least one active agent are administered into the body and their location monitored via, e.g. ultrasound, although additional monitoring may be achieved via one or more contrast agents encapsulated in the AALDS particles. When the AALDS particles have reached the desired target area, active agent release is then accomplished, preferably via AALDS particle rupture via ultrasound pulses.

Another embodiment of the present invention is directed to the use of the AALDSs of the present invention in the targeted delivery of active agent to one or more areas of interest of the body, where targeting specificity is enhanced by active targeting, i.e., by the use of modified AALDS system in which, e.g., AALDS particles are modified via ligands to direct the AALDS particles to a particular target.

One embodiment of the present invention is directed to a novel class of AALDS particles, contemplated to be smaller than conventional AALDS particles, i.e., of a few microns or less, and preferably less than about 1 μm.

### BRIEF DESCRIPTION OF THE DRAWINGS

FIG. 1a is a schematic diagram of the experiment and apparatus. A thin rubber tube was fixed in the water tank at a certain depth. After filling the tube with various liposome preparations at a concentration of 2 mg/mL, ultrasound images were recorded using a USI system connected with a ultrasound transducer (30 MHZ). Wash-in /wash-out software was used to analyze gray-scale imagings of liposomes.

FIG. 1b is a schematic diagram of the flow mode ultrasound imaging setup. The plastic tube was immersed under the water at room temperature. The ultrasound transducer was placed above it at a fixed position. During an experiment, 0.9% saline solution was infused into the tube at an approximate rate of 60 ml/min. A syringe was connected with the tube, and liposome solution was injected at an approximate rate of 1 ml/s.

FIG. 2, left and right panel, shows the size distribution profile of the liposomes before and after gas bubble formation in the intravascular space, respectively.

FIG. 3 shows the ultrasound images of liposomes in which the left panel is an image of the inventive liposme suspension without encapsulated gas bubbles, and the right panel is an image of the inventive liposome suspension containing gas bubbles, and the lower panel is time-intensity curve of the liposome images and without bubbles.

FIG. 4 is the time-intensity curve of the liposomal bubble formulations after injection, using a flow mode imaging set up illustrating increase in intensity approximately 10 seconds after the bolus injection of the bubbles and gradually reaching peak intensity (PI), with an enhancement from baseline to PI estimated of more than 20 DB.

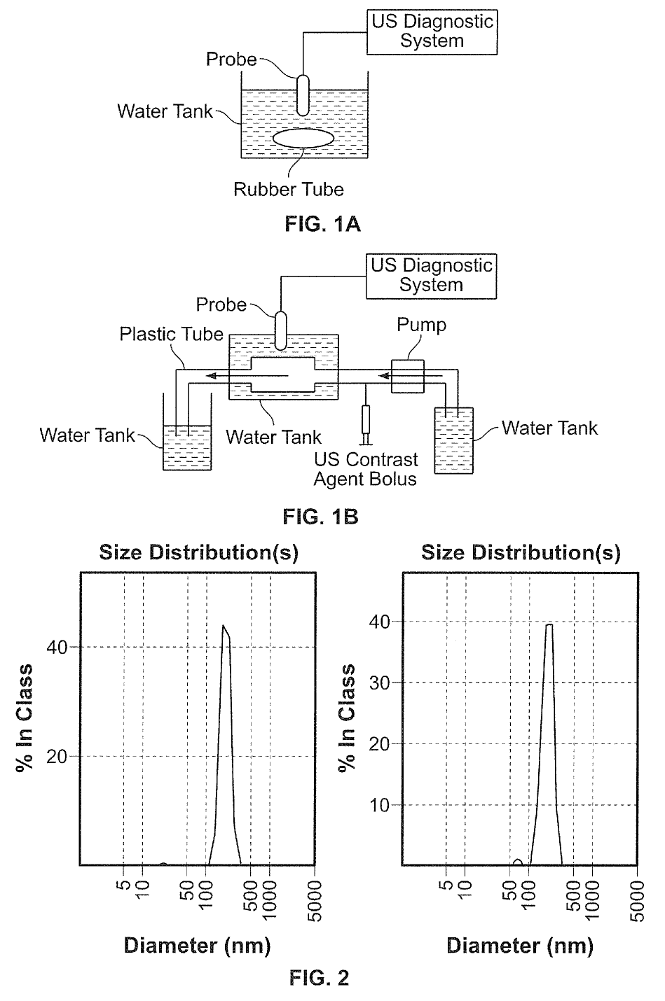

### DETAILED DESCRIPTION OF THE INVENTION

“Active agent” as used herein refers to any agent which is to be delivered into the body to exert an effect, including both diagnostic and therapeutic effects. Thus active agents include nucleic acids (RNA, DNA), proteins, small molecules, pharmaceuticals etc. Active agents include contrast agents such as gadolinium-based contrast agents. Exemplary active agents are provided as “biologically active substances” in U.S. Pat. No. 5,807,572, the contents of which are herein incorporated by reference in their entirety.

“Contrast agents” as defined herein refer to agents which improve the efficacy of imaging techniques such as USI and MRI. Two classes of contrast agents specifically contemplated are those based on air bubbles such as ALBUNEX™ and those based on Gadolinium (“Gd”), such as MAGNEVIST®.

“Active targeting” as defined herein refers to the targeting of AALDS particles to a particular region of the body, particular tissue type, and cells etc., via the use of targeting ligands, i.e., modifiers of the AALDS particles that result in the selective or preferential targeting of those particles to the region, tissue, and cells etc., desired. Examples of targeting ligands may be found in, for example, U.S. Pat. No. 6,521,211, the contents of which are herein incorporated by reference in their entirety.

“Intravesicular space” as defined herein refers to the space in the liposomes, lipid particles and vesicles, wherein the gases are formed or encapsulated.

Various embodiments of the invention include methods to introduce gaseous phases into liposomes and various other lipid based drug delivery systems. Particularly, the liposomes or LDSs may be those that would be preferably taken up by macrophages; those that could concentrate through the leaky neovasculatures and accumulate in tumors; those that incorporated an active targeting antibody (or ligand) and would bind to specific molecular target in the body; those that contain encapsulated or incorporated drugs; lipoplexes that were loaded with DNA, RNA or other nucleic acid based therapeutics; and combination of the above. The acoustically active LDSs can be used to imaging specific diseased site in the body, imaging specific molecular target in the body, to release drugs in a specific site in the body where the ulrasound is directed, and to release and deliver DNA to specific cells.

An embodiment of the invention is a new preparation method for gas containing liposome and lipid complex based on chemical reactions in intravesicular space. The gas formation processes are regulated by the lipid membrane composition and properties. Besides, further surface modification through the incorporation of targeting ligands will be beneficial for the specific in vivo delivery.

A method for preparation of gas containing liposomes and lipid complexes, comprising preparation of the liposome encapsulating one or more reactants, then adding the other reactants into the liposome suspension for diffusion through the lipid membrane to generate gas bubbles inside liposome.

Another embodiment of the invention is a method for preparation of gas containing liposomes and lipid complexes, comprising encapsulating insoluble gas forming precursors or soluble reactants that would react with time or upon light or heat stimulation.

In another embodiment, the reactants in the method are selected from the group consisting of hydrogen ion, nitrates, phosphates, carbonates, hydrochlorides, citrates, oxides, peroxides and their combinations. The reactants have a concentration range of about 0.001 mg/ml to about 150 mg/ml.

In another embodiment, the gas containing liposomes and lipid particles have a diameter of about 10 nm to about 10 μm. The lipid membrane can be unilamellar, multilamellar or arranged in a more complex morphologies, composed of at least one type of amphiphilic molecule, such as natural or synthesized phosphatidylcholine, glycolipids, sterols, fatty acid, surfactants, amphiphilic polymers and their combinations. Perferably, the membrane would contain one type of cationic lipids such as 1,2-Dioleoyloxy-3-trimethyl-ammonium-propane (DOTAP) or fatty acids such as lauric acid for improved membrane permeability of the outside reactants. The liposomes and lipid particles may also contain one or more therapeutic agents comprising drug substances, small chemical molecules, proteins, polypeptides, oligonucleosides, nuclear enzymes, DNA plasmids, and polymers, inside the intravesicular space, inside the membrane, or on the surface. The gas containing liposomes and lipid complexes may be further modified through physical, chemical and biological methods by linkage of hydrophilic polymers, ligands, antibodies, cytokines, polypeptides, nucleic acids and their combination to the surface of the liposomes and lipid complex to change the in vivo biodistribution of the liposomes and lipid complex.

The chemical reaction in intravesicular space involve preparation of liposome or lipoplexes encapsulating one or more reactants, for example, sodium carbonate; then adding the other reactants such as an acid, after removal of the solution sodium carbonate through dialysis. Preferably, the proton diffusion rate through the lipid membrane can be regulated by varying lipid compositions. Membranes that contain cationic lipids such as DOTAP or fatty acids such as lauric acid were found to have improved permeability of protons.

The carbon dioxide gas inside liposomes could be generated through another method: one or more water-insoluble reactant(s), for example calcium carbonate nanocrystals were encapsulated inside the intravesicular space of the liposomes first by methods such as core-shell particle synthesis. Then the liposome solution is dialyzed for solvent substitution. By adding acid solvent into the liposome suspension, hydrogen ion will diffuse through the membrane phase of the lipid particles to initiate carbon dioxide formation.

In another embodiment, reactants such as sodium carbonate and gluconolactone were both encapsulated in the aqueous phase inside lipid particles. They would react with water and each other to finally generate gas bubbles associated with the lipid particles.

The gas filled liposomes or lipid particles are stable under room temperature for 10 minutes to 12 hours. They are of a smaller size than conventional AALDS particles, i.e., of a few microns or less, and preferably less than about 1 μm.

One embodiment of the present invention is directed to a novel class of AALDS particles having strong echogenic properties and can be used as in vivo ultrasound contrast agents.

The other embodiment of the present invention is directed to a novel targeted drug/gene delivery system, with improved transfection efficiency and ultrasound directed site specificity.

Another embodiment of the present invention is a Computer Tomography (CT) contrast agent, with reduced CT density as a potential negative contrast agent for CT imaging.

Phosphatidylcholine (SPC), 1,2-dioleoyl-3-tri-methylammonium-propane (DOTAP) and cholesterol (CH) were mixed at specific ratios and dissolved in chloroform. The solvent was evaporated by using a rotary evaporator immersed in a thermostated water bath with the temperature at 35° C. The resulted lipid film was then hydrated using an aqueous solution containing NaHCO₃ (0.1 M). The flask was briefly sonicated using a water bath sonicator. The liposome suspension was then dialyzed against a large volume of 0.1 M NaCl solution for about 10 min, then 0.1 M citric acid solution was added into the suspension and incubated for several minutes. The solution's pH can then be adjusted back to 6 if needed.

The size distribution of liposomes was determined by photon correlation spectroscopy (PCS) using a Malvern 3000 system (Malvern Ltd, Malvern, UK). The values of the viscosity and refractive index used in the measurements were 0.8905 g/m and 1.333, respectively. After sonication, the average sizes of the liposomes formed were about 200 nm. The size distribution profile is shown in FIG. 2. After adding citric acid, CO₂ gas bubbles started to form in the intravesicular space of the liposomes. The liposome sizes did not change much. The polydispersity index maintained at less than 0.15, indicating a rather homogeneous particle population.

The gas bubble sizes and their distribution are considered essential for image enhancement quality with ultrasound. Most contrast agents currently available are in micrometer sizes. The micron particles would be confined inside the blood vessels after intravenous injection, therefore could be only used for vesicular imaging. The liposomal particles we obtained were much smaller. With sizes at 200 nm, they would be able to extravasate through the leaky neovasculature of a tumor, therefore enabling tumor imaging. Further more, it would be easy to use existing method to attach target specific ligands onto the liposome surface for molecular imaging.

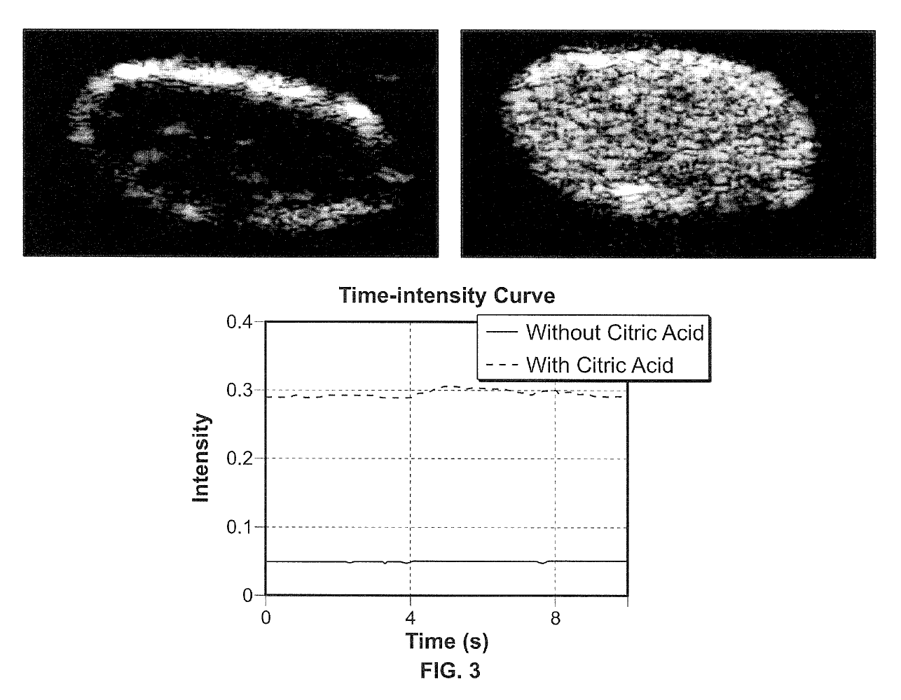

The echogenic properties of the liposomal bubble formations were evaluated by imaging the solutions in vitro in two different setups. FIG. 3 shows the low-MI grey-scale imaging in the stationary mode setup and the time-intensity curve of the solutions. With the formation of CO₂ gas bubbles inside liposomes, the ultrasound image intensities were greatly enhanced. The images are much brighter. The time-intensity curve showed that the intensity increased more than five times.

The measured intensity would usually not change within 40 min after adding citric acid into the liposome formulation. This indicates that the gas bubbles inside liposome were quite stable for at least 30 min.

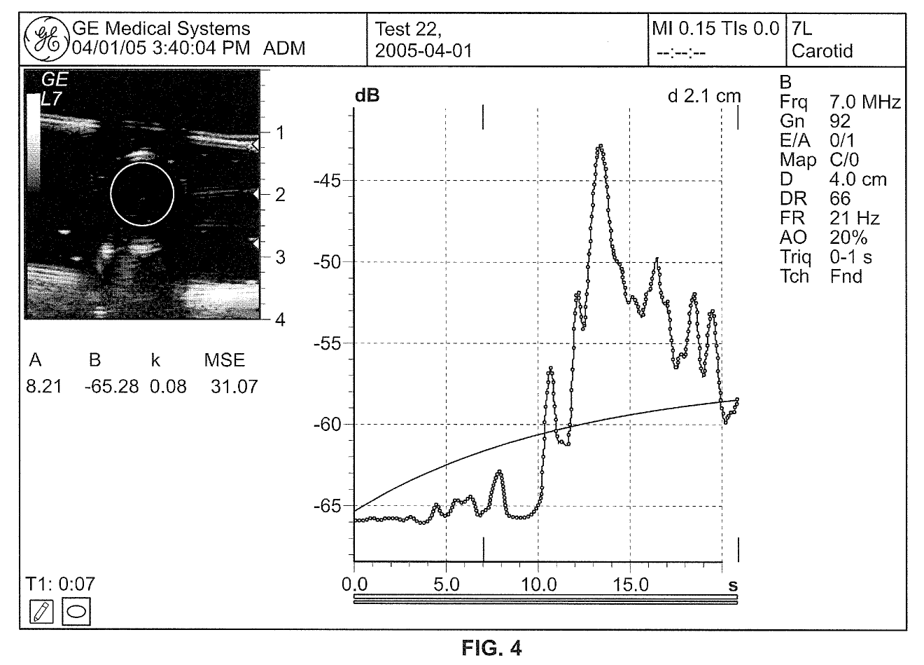

Using a flow mode imaging setup, the time-intensity curve of the liposomal bubble formulations after injection was obtained as shown in FIG. 4. The intensity began to increase about 10 seconds after bolus injection of the bubbles and gradually reached peak intensity (PI). The enhancement of the intensity from the baseline to PI was estimated to be more than 20 dB.

The formation kinetics of CO₂ gas bubbles in the intravesicular space of the liposomes were found to depend on several important parameters. The encapsulated NaHCO₃ concentration and the added citrate acid concentrations were both critical. Too high concentrations would result in too much and too fast CO₂ formation and disrupted lipid membranes. Too small concentrations would not produce enough gas inside the liposomes to form bubbles.

In addition, the lipid composition is important as well. Cationic lipids such as DOTAP were found to be essential to facilitate proton permeation through the membrane for the generation of gas bubble in the intravascular space. Huang had also reported that optimal acoustic stability was found with CH concentrations of 10-15 mol % (Huang et al., “Liposomes as ultrasound imaging contrast agents and as ultrasound-sensitive delivery agents,”Cellular & Molecular Biology Letters, vol 7, No. 2, pp. 233-235, 2002, which is incorporated here in its entirety by reference). Addition of cholesterol molecules in lipid membranes to affect the thickness and rigidity of the shell to obtain improved acoustic stability is proposed, as suggested by Calliada et al (Calliada et al., “Ultrasound contrast agents basic principles,” European Journal of Radiology, vol 27, pp. 157-160, 1998, which is incorporated here in its entirety by reference).

In another embodiment, acoustically active lipid/DNA complexes were prepared. Cationic liposome, which prepared by the methods described above, were mixed with DNA plasmids at a (±) charge ratio of 4:1. The mixture was incubated for 5 minutes in room temperature. 0.1M citric acid was added at 1:1 volume ratio of the liposomes. After 5 minutes, 20 μl NaOH—PBS was added to neutralize the solution to pH 7.

In vitro DNA transfection: SPCA-1 cells were harvested by trypsin treatment and suspended in cell medium. The cell number was adjusted by a cell counter and 1×10⁵ cells per well were plated on 24-well plates (Costar) at 37° C in an atmosphere of 5% CO₂ in RPMI 1640 supplemented with 10% Fetal Bovine Serum (FBS, Gibico) for 24 hours. Plasmid vector pGL3-control, which encoding the luciferase gene, was used as a reporter. Once the medium was removed, cells were washed with RPMI 1640 medium without FBS. The bubble containing complexes described above which contained 3 μg plasmids were added into each wells of the plate. And then the plate was placed on a chamber filled with 37° C. degassed water. A flat ultrasound transducer was placed on the bottom of the chamber. The cells were exposed in ultrasound from 0.5 min to 2 min and the ultrasound intensity ranged from 0.2 W/cm² to 0.8 W/cm². After ultrasound treatment, the lipoplexes containing medium was removed and FBS containing medium was added. Then the cells were incubated in 37° C. in an atmosphere of 5% CO₂ for 24 h. Transgene expressions were measured by luminometer 24 h after the transfection using luciferase assay kit and showed about 10-100 times increase over controls without ultrasound treatment.

In vivo transfection: Female BALb/c mice were anesthetized using 40 mg/ml chloral hydrate at 1% volume relative to body mass. After complete sedation, mice were given an injection of lacoustically active lipoplexes per gastrocnemius muscle, followed by focused ultrasound irradiation for 2 minutes. The working frequency of the ultrasound transducer was 1.0 MHz and the diameter of the transducer was 30 mm. The transducer was set up on the top of a columnar housing covered by latex. The columnar housing was filled with circulated degassed water to prevent overheating. The mice were sacrificed and the gastrocnemius muscles were examined for transgene expression. Studies also showed a significant improvement of gene transfection efficiency in vivo.

While the present invention has been described with reference to its preferred embodiments, one of one of ordinary skill in the relevant art will understand that the present invention is not intended to be limited by these preferred embodiments, and is instead contemplated to include all embodiments consistent with the spirit and scope of the present invention as defined by the appended claims. The entire disclosures of all references, applications, patents, and publications cited herein are hereby incorporated by reference.

What is claimed is:

1. A method for preparation of gas containing liposomes and lipid particles comprising preparing liposomes or lipid particles comprising a lipid membrane encapsulating a first reactant species and introducing a second reactant species by membrane controlled diffusion to generate gas bubbles inside the liposomes or lipid particles.

2. The method of claim 1, wherein the first and second reactant species are selected from the group consisting of hydrogen ion, nitrates, phosphates, carbonates, hydrochlorides, citrates, oxides, peroxides, gluconolactone and their combinations.

3. The method of claim 2, wherein the first and second reactant species are present at a concentration of about 0.001 mg/ml to about 150 mg/ml.

4. The method of claim 1, wherein the gas containing liposomes and lipid particles comprise a diameter of about 10 nm to about 10 μm.

5. The method of claim 1, wherein the liposomes or lipid particles are unilamellar, multilamellar, or multivesicular, having a lipid wall composed of at least one amphiphilic molecule, such as natural or synthesized phospholipids, glycolipids, sterols, fatty acids, surfactants, amphiphilic polymers and their combinations.

6. The method of claim 1, wherein the lipid membrane comprises at least one lipid selected from the group consisting of cationic lipids and fatty acids.

7. The method of claim 1 wherein the gas containing liposomes and lipid particles contain one or more therapeutic agents comprising drug substances, small chemical molecules, proteins, polypeptides, oligonucleosides, nuclear enzymes, DNA plasmids, and polymers, inside the intravesicular space, inside the membrane, or on the surface.

8. The method of claim 1 wherein the gas containing liposomes and lipid particles are further modified through physical, chemical and biological methods by linkage of hydrophilic polymers, ligands, antibodies, cytokines, polypeptides, nuclear acids and their combination to the surface of the liposomes and lipid particles to change the in vivo biodistribution of the liposomes and lipid particles.

9. A method for preparation of gas containing liposomes and lipid complexes, comprising the steps of encapsulating insoluble reactants, soluble reactants or both that would interact over time, upon light or heat stimulation inside the liposomes.

10. The method of claim 9, wherein the gas containing liposomes and lipid complexes comprise a diameter of about 10 nm to about 10 μm.

11. The method of claim 9, wherein the liposomes are unilamellar, multilamellar or multivesicular, having a lipid wall composed of at least one amphiphilic molecule, such as natural or synthesized phospholipids, glycolipids, sterols, fatty acids, surfactants, amphiphilic polymers and their combinations.

12. The method of claim 9 wherein the gas containing liposomes and lipid complexes contain one or more therapeutic agents comprising drug substances, small chemical molecules, proteins, polypeptides, oligonucleosides, nuclear enzymes, DNA plasmids, and polymers, inside the intravesicular space or the surface.

13. The method of claim 9 wherein the gas containing liposomes and lipid complexes are further modified through physical, chemical and biological methods by linkage of hydrophilic polymers, ligands, antibodies, cytokines, polypeptides, nuclear acids and their combination to the surface of the liposomes and lipid complex to change the in vivo biodistribution of the liposomes and lipid complex.

14. The acoustically active liposomal particles prepared by the method of claim 1.

15. The acoustically active liposomal particles of claim 14, having a maximum particle dimension of about 5 μm or less.

16. The acoustically active liposomal particles prepared by the method of claim 9.

17. The acoustically active liposomal particles of claim 16, having a maximum particle dimension of about 5 μm or less.
